(19) United States
(12) Patent Application Publication
(10) Pub. No.: US 2009/0053806 A1
(43) Pub. Date: Feb. 26, 2009

(54) COATING SOLUTION AND METHOD FOR CAPTURING AND PRESERVING BIOLOGICAL MATERIALS

(76) Inventors: Deirdre Daniels, Shawnee, KS (US); Darren Radke, Overland Park, KS (US)

Correspondence Address: HUSCH BLACKWELL SANDERS LLP, 4801 Main Street, Suite 1000, KANSAS CITY, MO, 64112, US

(21) Appl. No.: 11/843,441

(22) Filed: Aug. 22, 2007

(51) Int. Cl. C12N 5/06

(52) U.S. Cl. 435/374; 435/404

(57) ABSTRACT

The present invention is directed to a coating solution for capturing and preserving biological materials. The coating solution includes at least one saccharide. The coating solution may also include at least one other constituent, such as amino acids, complex proteins, surfactants, and mixtures thereof. The present invention is also directed to providing a method for collecting and preserving biological material using a coating solution.

### STATEMENT REGARDING FEDERALLY SPONSORED RESEARCH OR DEVELOPMENT

The invention described herein was partially supported by the government of the United States of America. The invention described herein may be manufactured and used by or for the government of the United States of America for government purposes and the government has certain rights in the invention.

### BACKGROUND OF THE INVENTION

Bioterrorism, the intentional release or dissemination of biological agents, especially Bacillus anthracis (the causative agent of anthrax) and other hazardous bacteria, viruses, venom or toxins, has become a major concern. Bioterrorist attacks are, by their nature, difficult to prevent and identify. One concern is that these types of materials will be released in crowded areas such as airports and on airplanes in an effort to infect and terrorize a great number of people at once. In a situation where a biological material has been released, it is imperative that the exact pathogen released be quickly identified to reduce the number of severe casualties. Government agencies, including the Department of Defense, Federal Bureau of Investigation and Department of Homeland Security, as well as private industry, are expending considerable resources toward researching and developing means of preventing, identifying, and ameliorating bioterrorist attacks. These government agencies and private contractors are also involved in the detection, collection and analysis of biological materials.

A variety of methods and types of equipment are used to collect biological materials whether the materials are airborne or visible solids. In order to analyze a particular biological material, a sample must be collected, secured, and transported to a laboratory for analysis. During transport, the sample may significantly destabilize or decrease in size thereby making it difficult to analyze and/or make an accurate determination of the biological materials involved. The more time that passes, the more difficult it becomes to remedy any damage and effectively clean and contain the contaminated area.

In order to effectively collect the biological material, one common method is to use a liquid solution within a collection device to capture the sample and aid in effectively removing the sample from the collection device. The liquid solution is then removed from the collection device and the sample is analyzed. Several disadvantages associated with using a liquid solution as a capturing method include that the liquid increases the weight of the collection device, is susceptible to temperature fluxes leading to evaporation or freezing of the sample, and has the potential to be spilled. Therefore, it would be beneficial for a collection and preservation solution to be lightweight, temperature insensitive and not susceptible to spillage.

Another common type of collection device, a biological impactor, does not use a liquid when collecting the biological material, but rather collects an air sample and then impacts it onto a surface, usually an agar. Although it is advantageous to be able to transfer a collected sample directly to the incubator for analysis, there are also many disadvantages of using agar collectors. Agar is susceptible to temperature extremes, can easily become contaminated, can dry out quickly and has a preferred long-term storage condition of 2-8° C. These types of restrictions make field sampling difficult. Many biological impactors also require the agar to reside in glass Petri dishes which are fragile and not disposable. Therefore, it would be beneficial to have a collection and preservation solution that would not require special storage conditions after collection of a biological material and would also be easy to separate from the collection device in the laboratory.

### SUMMARY OF THE INVENTION

In one of many illustrative, non-limiting aspects of the present invention, there is provided a coating solution for capturing and preserving biological materials. The coating solution includes at least one saccharide, typically trehalose. The coating solution may also include at least one other constituent, including but not limited to, amino acids, complex proteins and surfactants.

In another of many illustrative, non-limiting aspects of the present invention, there is provided a method for collecting and preserving biological material using a coating solution. The method includes forming a coating solution including at least one saccharide that may be combined with at least one other constituent such as amino acids, complex proteins and surfactants; applying the coating solution to a container; curing the coating solution; adding a biological material to the container; dissolving the coating solution and the biological material in a liquid buffer; and removing the coating solution and the biological material for analysis.

### BRIEF DESCRIPTION OF THE SEVERAL VIEWS OF THE DRAWING

In the accompanying drawings that form a part of the specification and that are to be read in conjunction therewith:

FIG. 1 is a graphical representation of the recovery of Bacillus globigii from trehalose-coated tubes;

FIG. 2 is a graphical representation of the recovery of Bacillus globigii from trehalose/betaine-coated tubes;

FIG. 3 is a graphical representation of the recovery of Bacillus globigii from sucrose-coated tubes;

FIG. 4 is a graphical representation of the recovery of Bacillus globigii from sucrose/betaine-coated tubes;

FIG. 5 is a graphical representation of the recovery of Bacillus globigii from all four coatings;

FIG. 6 is a graphical representation of the recovery of Erwinia herbicola from trehalose-coated tubes;

FIG. 7 is a graphical representation of the recovery of Erwinia herbicola from trehalose/betaine-coated tubes;

FIG. 8 is a graphical representation of the recovery of Erwinia herbicola from sucrose-coated tubes;

FIG. 9 is a graphical representation of the recovery of Erwinia herbicola from sucrose/betaine-coated tubes;

FIG. 10 is a graphical representation of the recovery of Erwinia herbicola from all four coatings;

FIG. 11 is a graphical representation of the recovery of MS2 from trehalose-coated tubes;

FIG. 12 is a graphical representation of the recovery of MS2 from trehalose/betaine-coated tubes;

FIG. 13 is a graphical representation of the recovery of MS2 from sucrose-coated tubes;

FIG. 14 is a graphical representation of the recovery of MS2 from sucrose/betaine-coated tubes;

FIG. 15 is a graphical representation of the recovery of MS2 from all four coatings;

FIG. 16 is a graphical representation of the recovery of ovalbumin from trehalose-coated tubes;

FIG. 17 is a graphical representation of the recovery of ovalbumin from trehalose/betaine-coated tubes;

FIG. 18 is a graphical representation of the recovery of ovalbumin from sucrose-coated tubes;

FIG. 19 is a graphical representation of the recovery of ovalbumin from sucrose/betaine-coated tubes; and

FIG. 20 is a graphical representation of the recovery of ovalbumin from all four coatings.

### DETAILED DESCRIPTION OF THE INVENTION

There is provided herein a coating solution for the capture and preservation of biological material. A method for applying the coating solution to a collection device that collects biological material is also provided. The coating solution of the present invention is easy to prepare and use, does not add significant weight to the collection device, is substantially temperature insensitive and is easy to transport and use in the field. After curing, the non-liquid coating solution hereby is adaptable for use with a variety of collection devices, including but not limited to electrostatic precipitators, biological impactors, dry cyclones, swabs, wipes, and filters. The coating solution can therefore be used in a collection device that is used for the dry capture of biological materials in the field.

The coating hereof includes at least one saccharide. In an illustrative example, the coating solution preferably includes from about 100 mM to 300 mM, more preferably from about 150 mM to 250 mM and, most preferably about 200 mM, of at least one saccharide. Illustrative saccharides include, but are not limited to, glucose, fructose, galactose, ribose, trehalose, sucrose, lactose, maltose, cellobiose, raffinose, hydrophilic polysaccharides, and mixtures thereof. In one embodiment of the present invention, trehalose is particularly preferred for use in the present coating solution.

The coating solution may also include at least one other constituent. In an illustrative example, the coating solution may include from about 0.1 mM to 30 mM, more preferably from about 1 mM to 10 mM and, most preferably about 2 mM, of one or more amino acids, complex proteins, surfactants and mixtures thereof. Amino acids that may be used in the coating solution hereof include, but are not limited to, betaine (also known as trimethylglycine) alanine, arginine, asparginine, aspartic acid, cysteine, glutamic acid, glutamine, glycine, histidine, isoleucine, leucine, lysine, methionine, phenylalanine, proline, serine, threonine, tryptophan, tyrosine, valine, mixtures thereof and derivatives thereof. Complex proteins that may be used in the coating solution hereof include, but are not limited to, proteose peptones, peptone, casitone, tryptone, tryptose, soytone, beef extract, yeast extract, malt extract, casein, soybean casein digest, eggs (yolks and/or whites), fish peptone, milk, infusion from muscle, brains, heart, or liver, serums, albumins, mixtures thereof and derivatives thereof. Surfactants that may be used in the coating solution hereof include, but are not limited to, Tween® products, Triton® products, Brij® products and other suitable non-ionic surfactants. In one embodiment of the present invention, betaine is used in combination with the saccharide described hereinabove in the coating solution. In another illustrative embodiment, the coating solution includes 200 mM of trehalose with 2 mM of betaine.

The coating solution should be made immediately prior to coating a collection container that will be used in the collection device. The following is an illustrative example of one method (a dip coating method) of applying the coating solution to the collection container, e.g. a tube. The tube should be thoroughly cleaned and dried and then sealed in an airtight container prior to being coated to ensure the integrity of the tube if not being coated immediately. One end of the tube is capped leaving the other end of the tube open. The tube is filled with the coating solution to ¼ of the length of the tube by pouring the coating solution into the tube. An alternative to pouring straight into the tube is to use a pipette to add the coating solution to the tube which helps reduce bubbles. The open end of the tube is then capped and the tube is rotated gently in a horizontal position thereby ensuring that the coating solution coats the entire surface of the inside of the tube. Excess coating solution is then poured out. The extra coating solution may be saved and used to coat more tubes. The cap from the other end is removed and the tube is positioned upright on a paper towel or cotton towel for approximately five minutes to allow any excess liquid to drain. The tube may be cured at a temperature of from about room temperature (59-86° F.) up to about 180° F. An illustrative example of the method for curing the coating solution is to place the tube in a vacuum oven at a setting of approximately 20 in. Hg vacuum and 100° F. for 1½ hours to remove any water content. The tube may then be sealed in an airtight container for storage prior to being added to the collection container.

Surfaces of the collection container, usually but not limited to interior surfaces, may be pre-treated prior to application of the coating solution by treating and/or prepping the surfaces using a chemical, mechanical, or electrical treatment depending on the material of the surfaces. The coating solution may be applied to a variety of surfaces and materials found in the collection device, including, but not limited to, aluminum, other metals, alloys, glass, filters made of a variety of materials, swabs made of a variety of materials, raw surfaces, plastics, composite laminates, including fiberglass, graphite, and Kevlar®, painted or treated surfaces, pores and semi-pores materials or other appropriate surfaces now known or hereafter developed.

The dip coating method described above is one example of the method that may be used to coat the collection container or even the collection device with the coating solution. Alternatively, the coating solution may be sprayed on, wiped on, painted on, or brushed on to the inside surfaces of the collection container and then cured using the vacuum oven as previously described. Depending on the type of coating method used and the material to which the coating is being applied, one or more additional applications of coating solution may be desired.

Once the collection container is coated, the biological material is added to the container either in the field or in a laboratory. Once the biological material has been added, it is necessary to remove the biological material and the coating solution from the collection container in order to analyze the material. This may be accomplished by using a liquid buffer in which the saccharide-based coating solution and any additional constituents would dissolve. Illustrative liquid buffers include, but are not limited to, from about 0.01-1.0% of water, water-containing salts, surfactants, complex proteins, or any combination thereof. In an illustrative example, the liquid buffer preferably includes from about 0.1% to about 1.0% NaCl and from about 0.01% to about 0.5% Triton X-100 and, most preferably about 0.5% NaCl and about 0.1% Triton X-100. Once the buffer has dissolved the coating solution and the biological material, the biological material may be analyzed using any appropriate methodology and/or apparatus including viable culture techniques, nucleic acid analysis, or immunoassay. The surfaces that were coated by the coating solution may be cleaned, re-coated, and re-used.

### EXAMPLES

The following examples are offered by way of illustration and not by way of limitation. It will be appreciated by those of ordinary skill in the art that any of the apparatus used herein may be substituted with other apparatus suitable for use in the methods described. Four different coating solutions were evaluated with biological material targets. The targets tested were Bacillus globigii (spore), Erwinia herbicola (vegetative cell), MS2 bacteriophage (virus), and ovalbumin (protein). The collection containers were aluminum tubes. The tubes were coated using the dip coating method described hereinabove.

In each of Examples 1-4, coated tubes were spiked separately with each target in triplicate. Targets were recovered from one set of coated tubes immediately and recovered from the other set 12 hours after spiking. Coated tubes were spiked at a concentration of at least 1e6 CFU/mL (spores and vegetative cells), 1e7 PFU/mL (virus) and 30,000 ng/mL (protein). The liquid inoculum was spread across the inner surface of the coated tubes and allowed to dry. Targets were recovered from all tubes by rinsing the coated tubes with 10 mL of recovery buffer (0.5% NaCl with 0.1% Triton X-100, pH 7) and recovering the rinse fluid into a 50-mL tube.

### Example 1

In Example 1, spores were analyzed using viable culture techniques. Recovered samples were diluted ten-fold in recovery buffer down to the desired concentration expected to contain 30-300 CFU/mL. One hundred microliters of the desired dilution were plated onto Trypticase Soy Agar (TSA) in triplicate and then incubated overnight at 35±2° C. and colonies counted to determine the concentration of spores or vegetative cells recovered.

### Example 1

### Results—Bacillus globigii

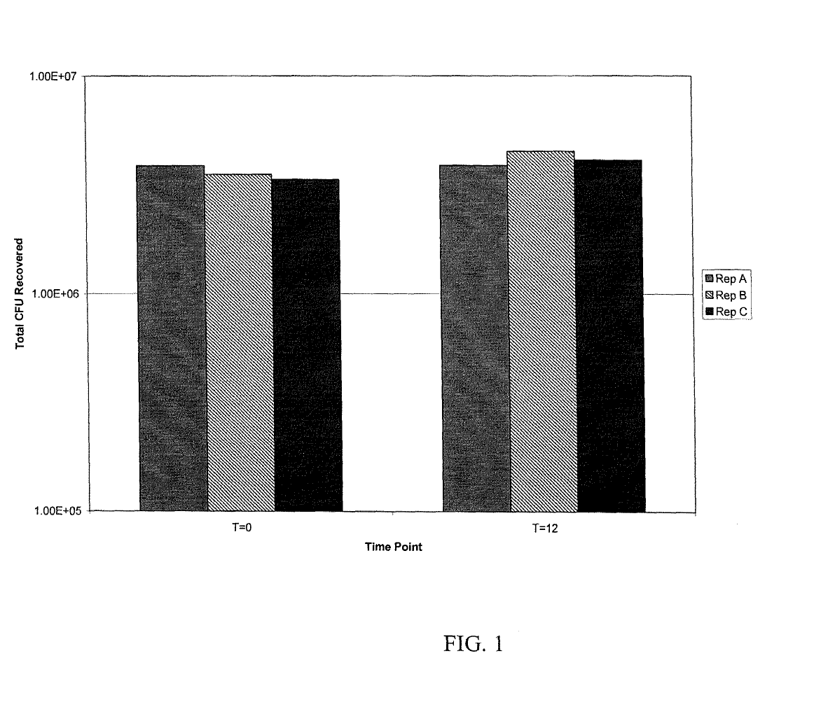

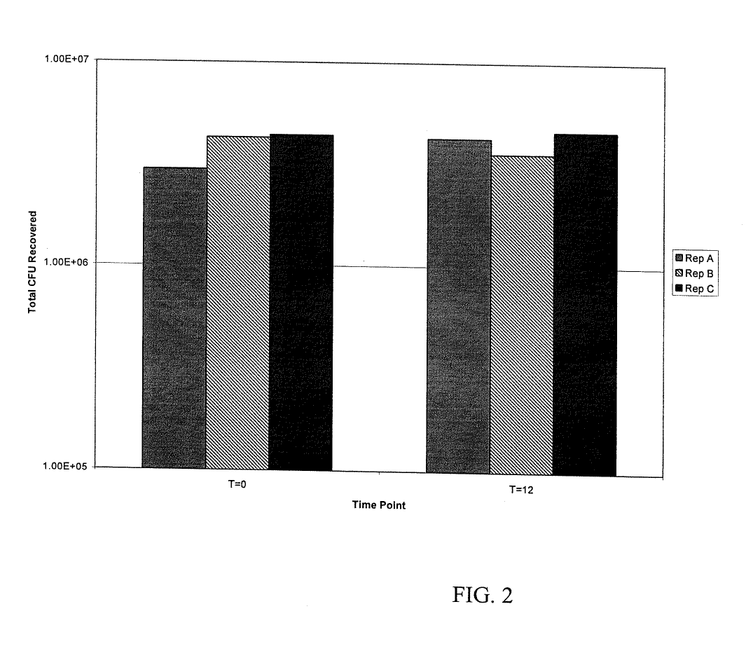

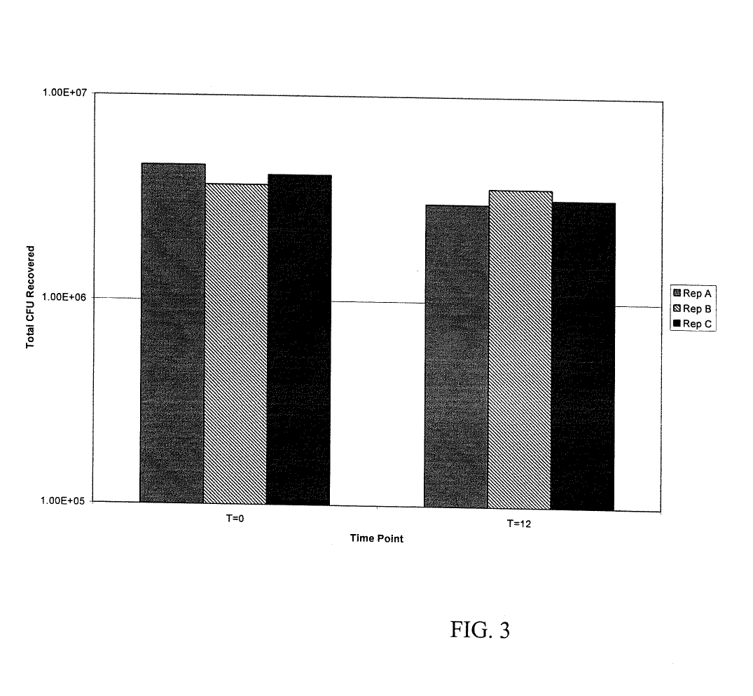

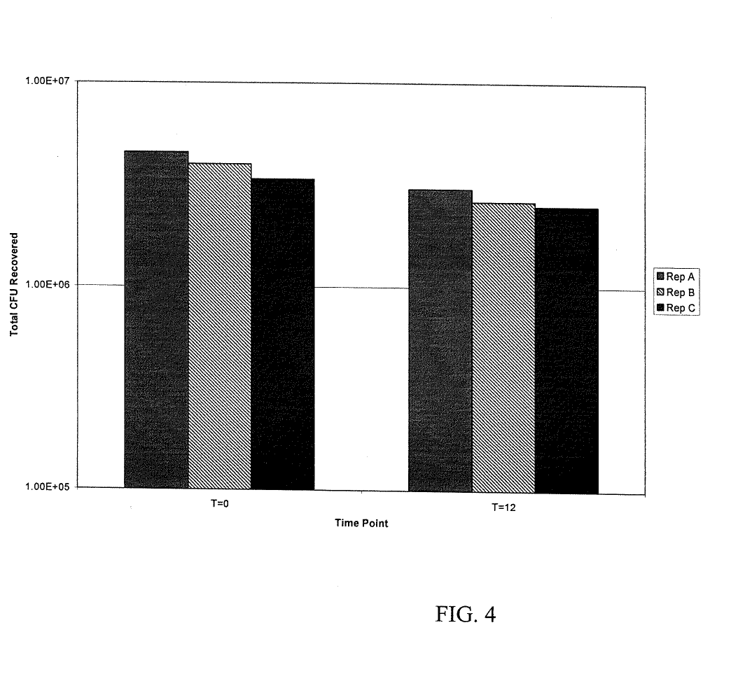

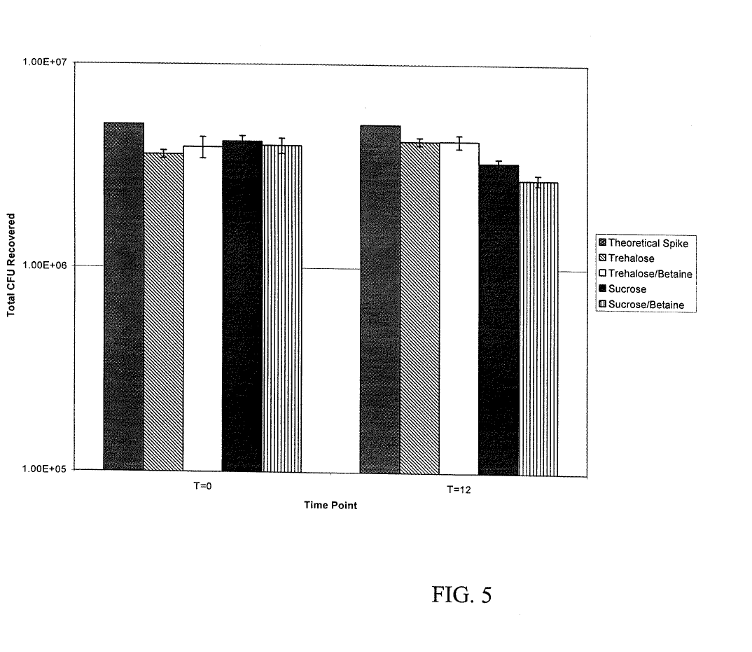

The results for all samples tested using Bacillus globigii (Bg) can be seen in Table 1 and FIGS. 1-5. Table 1 presents the raw data counts as seen on the agar plates and also shows the calculated total spore recovery from each sample. FIGS. 1 through 4 compare the recoveries at each time point for each coating separately. According to these figures, recovery of spores appears to be consistent and reproducible regardless of time point or coating. FIG. 5 summarizes FIGS. 1 through 4 and compares the spore recovery to the theoretical amount spiked onto the samples. According to FIG. 5, there appears to be no difference between the four coatings for recovery of spores. There may have been a slight loss in recovery of spores from the sucrose coatings at T=12 hours.

TABLE 1
Raw Data - Bacillus globigii

| | | | Dilution Plated | | | | | | | | | | |
|---|---|---|---|---|---|---|---|---|---|---|---|---|---|
| | | | 10⁻¹ | | | 10⁻² | | | 10⁻³ | | | | Total CFU |
| TimePoint | Coating | Replicate | A | B | C | A | B | C | A | B | C | Avg | Recovered |
| 0 | T | A | NP | NP | NP | NP | NP | NP | 39 | 35 | 42 | 39 | 3.87E+06 |
| | | B | NP | NP | NP | NP | NP | NP | 35 | 30 | 41 | 35 | 3.53E+06 |
| | | C | NP | NP | NP | NP | NP | NP | 41 | 32 | 27 | 33 | 3.33E+06 |
| | T + B | A | NP | NP | NP | NP | NP | NP | 27 | 31 | 31 | 30 | 2.97E+06 |
| | | B | NP | NP | NP | NP | NP | NP | 44 | 37 | 47 | 43 | 4.27E+06 |
| | | C | NP | NP | NP | NP | NP | NP | 42 | 40 | 50 | 44 | 4.40E+06 |
| | S | A | NP | NP | NP | NP | NP | NP | 49 | 53 | 36 | 46 | 4.60E+06 |
| | | B | NP | NP | NP | NP | NP | NP | 35 | 41 | 35 | 37 | 3.70E+06 |
| | | C | NP | NP | NP | NP | NP | NP | 25 | 53 | 46 | 41 | 4.13E+06 |
| | S + B | A | NP | NP | NP | NP | NP | NP | 52 | 42 | 42 | 45 | 4.53E+06 |
| | | B | NP | NP | NP | NP | NP | NP | 38 | 33 | 48 | 40 | 3.97E+06 |
| | | C | NP | NP | NP | NP | NP | NP | 43 | 33 | 25 | 34 | 3.37E+06 |
| 12 | T | A | NP | NP | NP | NP | NP | NP | 36 | 39 | 41 | 39 | 3.87E+06 |
| | | B | NP | NP | NP | NP | NP | NP | 47 | 47 | 41 | 45 | 4.50E+06 |
| | | C | NP | NP | NP | NP | NP | NP | 32 | 29 | 62 | 41 | 4.10E+06 |
| | T + B | A | NP | NP | NP | NP | NP | NP | 44 | 42 | 42 | 43 | 4.27E+06 |
| | | B | NP | NP | NP | NP | NP | NP | 35 | 40 | 33 | 36 | 3.60E+06 |
| | | C | NP | NP | NP | NP | NP | NP | 31 | 49 | 59 | 46 | 4.63E+06 |
| | S | A | NP | NP | NP | NP | NP | NP | 31 | 29 | 31 | 30 | 3.03E+06 |
| | | B | NP | NP | NP | NP | NP | NP | 36 | 30 | 42 | 36 | 3.60E+06 |
| | | C | NP | NP | NP | NP | NP | NP | 33 | 40 | 23 | 32 | 3.20E+06 |
| | S + B | A | NP | NP | NP | NP | NP | NP | 33 | 30 | 28 | 30 | 3.03E+06 |
| | | B | NP | NP | NP | NP | NP | NP | 30 | 22 | 27 | 26 | 2.63E+06 |
| | | C | NP | NP | NP | NP | NP | NP | 18 | 25 | 32 | 25 | 2.50E+06 |

KEY:
T = 200 mM trehalose
T + B = 200 mM trehalose/2 mM betaine
S = 200 mM sucrose
S + B = 200 mM sucrose/2 mM betaine
NP = Not Plated

### Example 2

In Example 2, vegetative cells were analyzed using viable culture techniques. Recovered samples were diluted ten-fold in recovery buffer down to the desired concentration expected to contain 30-300 CFU/mL. One hundred microliters of the desired dilution were plated onto Trypticase Soy Agar (TSA) in triplicate and then incubated overnight at 35±2° C. and colonies counted to determine the concentration of spores or vegetative cells recovered.

### Example 2

### Results—Erwinia herbicola

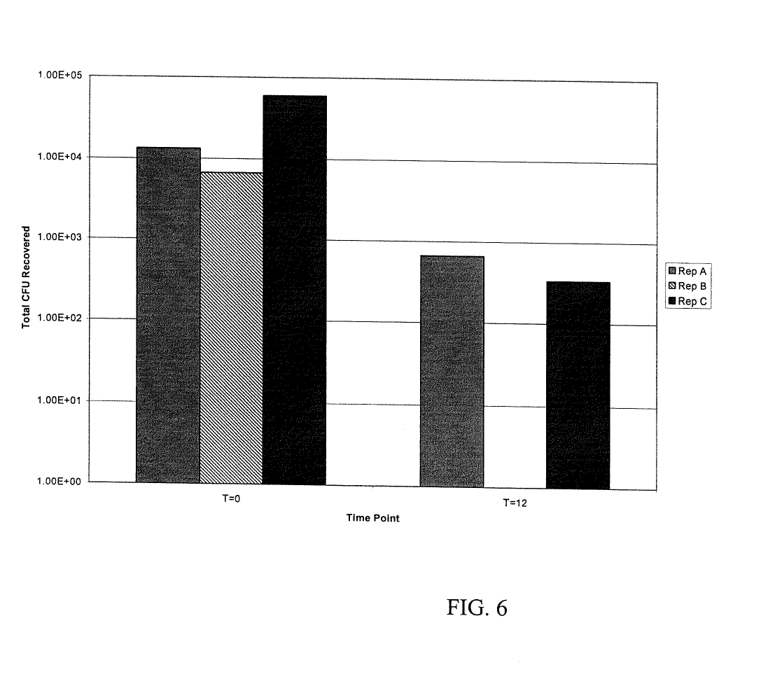

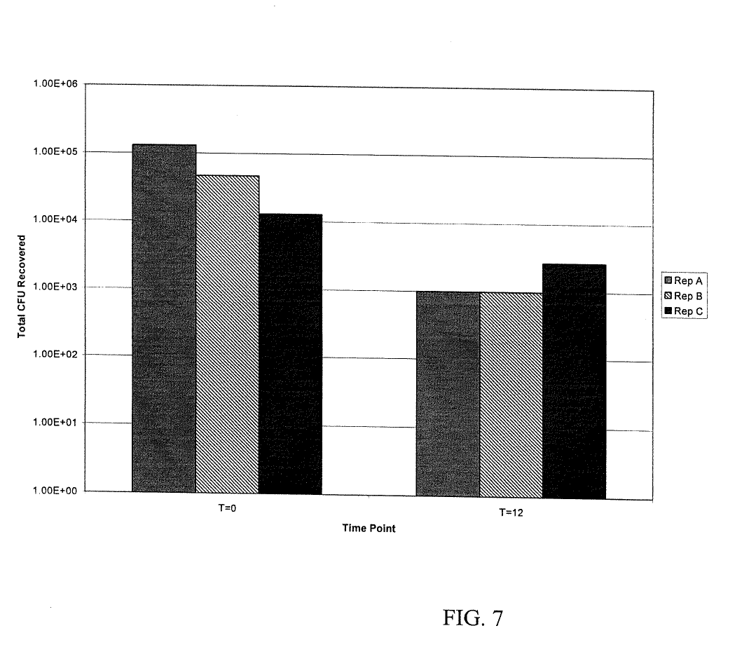

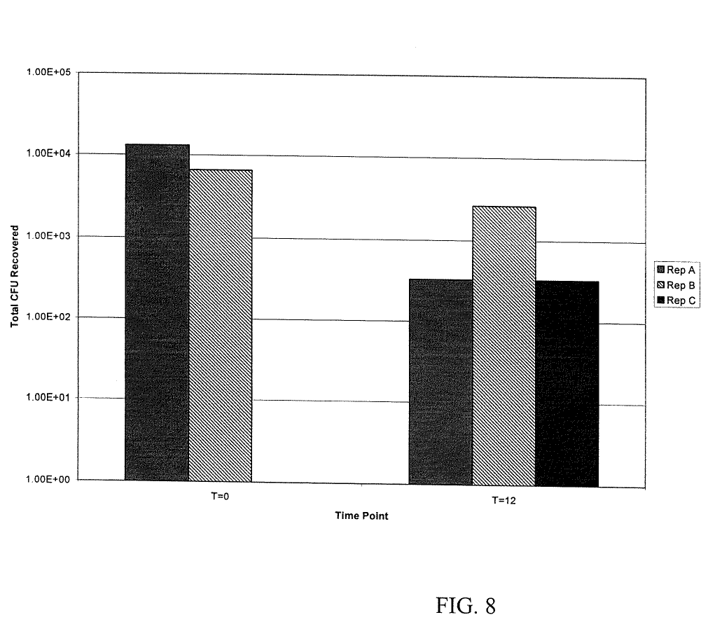

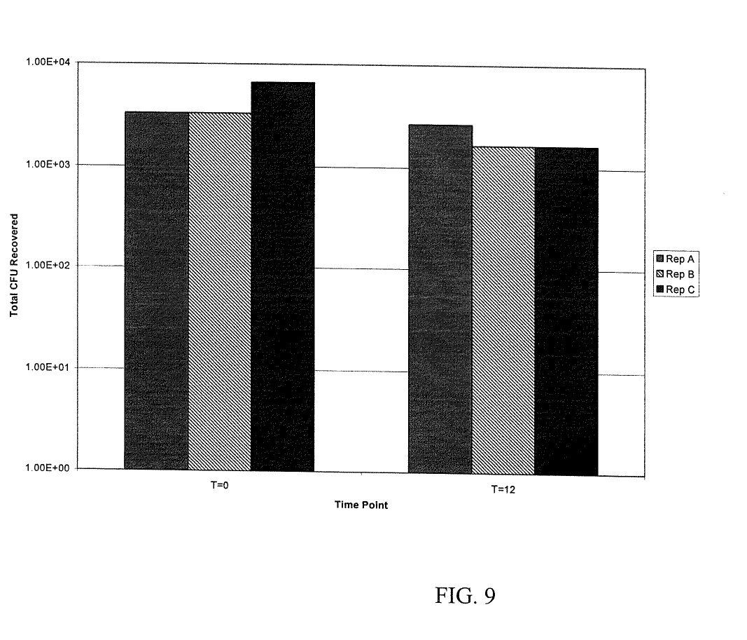

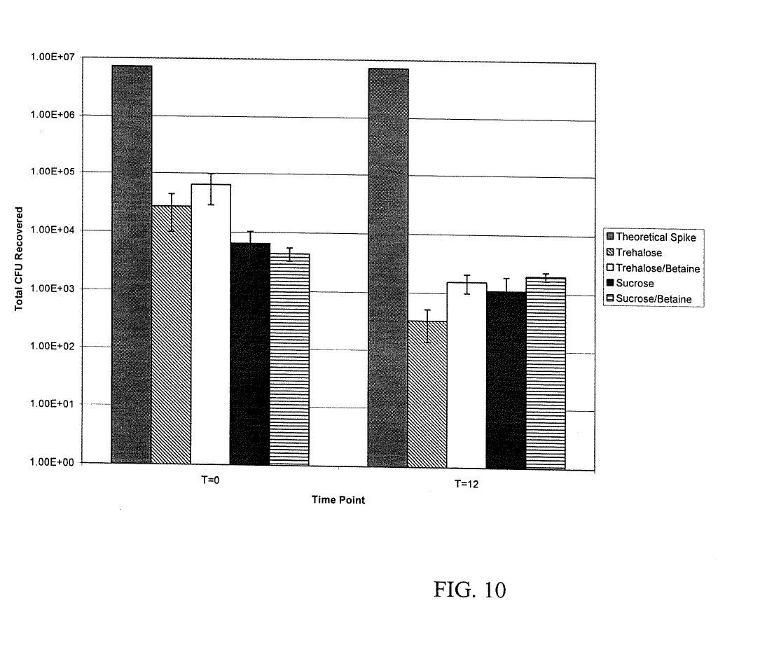

The results for all samples tested using Erwinia herbicola (Eh) can be seen in Table 2 and FIGS. 6-10. Table 2 presents the raw data counts as seen on the agar plates and also shows the calculated total vegetative cell recovery from each sample. The raw data indicates very poor viable recovery of vegetative cells from all of the samples. The ideal range for plate counts is 30-300 colonies and all counts fell below this value. The recovered samples were re-plated at lower dilutions, but the cells were no longer viable. FIGS. 6 through 9 compare the recoveries at each time point for each coating separately. These figures are based on data that fell below the 30-300 colony range and therefore further emphasize the poor and inconsistent viable recoveries from the replicates regardless of the coating. FIG. 10 summarizes FIGS. 6 through 9 and compares the vegetative cell recovery to the theoretical amount spiked onto the samples. According to FIG. 10, there is an immediate two to three log loss upon drying of vegetative cells prior to recovery at T=0. Vegetative cells are susceptible to desiccation and this result is somewhat expected. FIG. 10 also shows that even more loss in viable vegetative cell recovery occurs after 12 hours. It also appears that there was slightly better recovery of viable vegetative cells at T=0 from the tubes coated with trehalose solutions than those coated with sucrose solutions.

Although recovery of viable vegetative cells was poor, the samples were recovered in a buffer that would be compatible with nucleic acid extraction methods and PCR (polymerase chain reaction) analysis. Subjecting recovered samples to nucleic acid extraction and subsequent PCR would provide valuable genomic data regardless of viability.

TABLE 2
Raw Data - Erwinia herbicola

| | | | Dilution Plated | | | | | | | | | | |
|---|---|---|---|---|---|---|---|---|---|---|---|---|---|
| | | | 10⁻¹ | | | 10⁻² | | | 10⁻³ | | | | Total CFU |
| TimePoint | Coating | Replicate | A | B | C | A | B | C | A | B | C | Avg | Recovered |
| 0 | T | A | NP | NP | NP | 1 | 1 | 2 | 0 | 0 | 0 | 1.33 | 1.33E+04 |
| | | B | NP | NP | NP | 1 | 1 | 0 | 0 | 0 | 0 | 0.67 | 6.67E+03 |
| | | C | NP | NP | NP | 3 | 9 | 6 | 0 | 1 | 2 | 6.00 | 6.00E+04 |
| | T + B | A | NP | NP | NP | 12 | 16 | 11 | 1 | 2 | 0 | 13.00 | 1.30E+05 |
| | | B | NP | NP | NP | 3 | 7 | 4 | 2 | 0 | 0 | 4.67 | 4.67E+04 |
| | | C | NP | NP | NP | 3 | 1 | 0 | 0 | 1 | 0 | 1.33 | 1.33E+04 |
| | S | A | NP | NP | NP | 1 | 2 | 1 | 1 | 1 | 1 | 1.33 | 1.33E+04 |
| | | B | NP | NP | NP | 0 | 2 | 0 | 0 | 0 | 0 | 0.67 | 6.67E+03 |
| | | C | NP | NP | NP | 0 | 0 | 0 | 0 | 0 | 0 | 0.00 | 0.00E+00 |
| | S + B | A | NP | NP | NP | 1 | 0 | 0 | 0 | 0 | 0 | 0.33 | 3.33E+03 |
| | | B | NP | NP | NP | 1 | 0 | 0 | 0 | 0 | 0 | 0.33 | 3.33E+03 |
| | | C | NP | NP | NP | 0 | 1 | 1 | 0 | 0 | 0 | 0.67 | 6.67E+03 |
| 12 | T | A | 0 | 1 | 1 | NG | NG | NG | NG | NG | NG | 0.67 | 6.67E+02 |
| | | B | 0 | 0 | 0 | NG | NG | NG | NG | NG | NG | 0.00 | 0.00E+00 |
| | | C | 1 | 0 | 0 | NG | NG | NG | NG | NG | NG | 0.33 | 3.33E+02 |
| | T + B | A | 1 | 2 | 0 | NG | NG | NG | NG | NG | NG | 1.00 | 1.00E+03 |
| | | B | 2 | 0 | 7 | NG | NG | NG | NG | NG | NG | 1.00 | 1.00E+03 |
| | | C | 2 | 2 | 4 | NG | NG | NG | NG | NG | NG | 2.67 | 2.67E+03 |
| | S | A | 0 | 0 | 1 | NG | NG | NG | NG | NG | NG | 0.33 | 3.33E+02 |
| | | B | 3 | 5 | 0 | NG | NG | NG | NG | NG | NG | 2.67 | 2.67E+03 |
| | | C | 1 | 0 | 0 | NG | NG | NG | NG | NG | NG | 0.33 | 3.33E+02 |
| | S + B | A | 3 | 3 | 2 | NG | NG | NG | NG | NG | NG | 2.67 | 2.67E+03 |
| | | B | 1 | 2 | 2 | NG | NG | NG | NG | NG | NG | 1.67 | 1.67E+03 |
| | | C | 1 | 0 | 4 | NG | NG | NG | NG | NG | NG | 1.67 | 1.67E+03 |

T = 200 mM trehalose
T + B = 200 mM trehalose/2 mM betaine
S = 200 mM sucrose
S + B = 200 mM sucrose/2 mM betaine
NP = Not Plated
NG = No Growth

### Example 3

In this example, MS2 was analyzed using viable culture techniques. Recovered samples were diluted ten-fold in recovery buffer down to the desired concentration expected to contain 30-300 PFU/mL. Two hundred microliters of the desired dilution were inoculated into 10 mL of overlay agar (tempered to approximately 56° C. in a water bath) containing 200 μL of E. coli host and then poured into a standard Petri dish. Plates were prepared in triplicate and were incubated overnight at 35±2° C. and plaques counted to determine the recovered concentration of MS2.

### Example 3

### Results—MS2

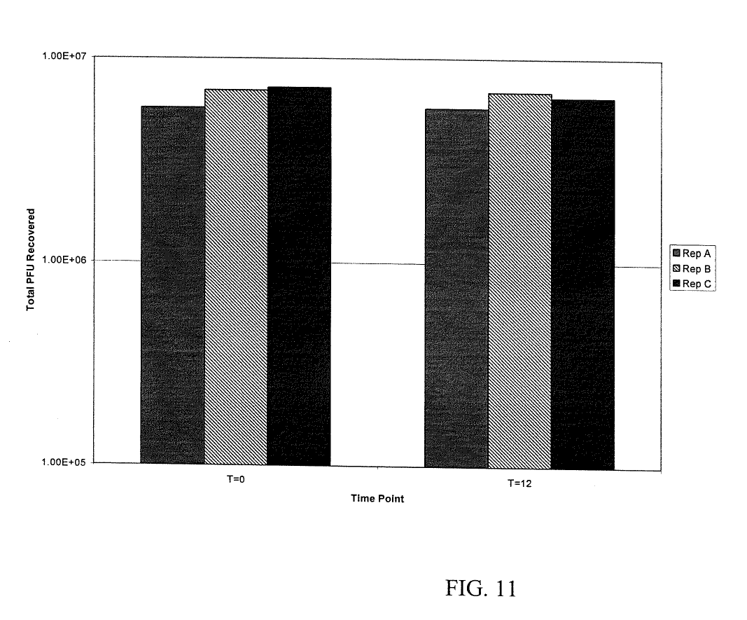

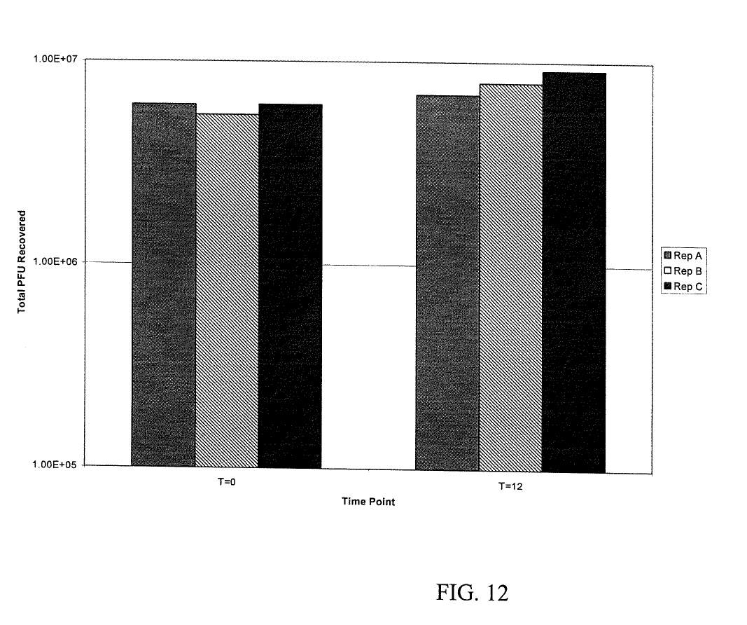

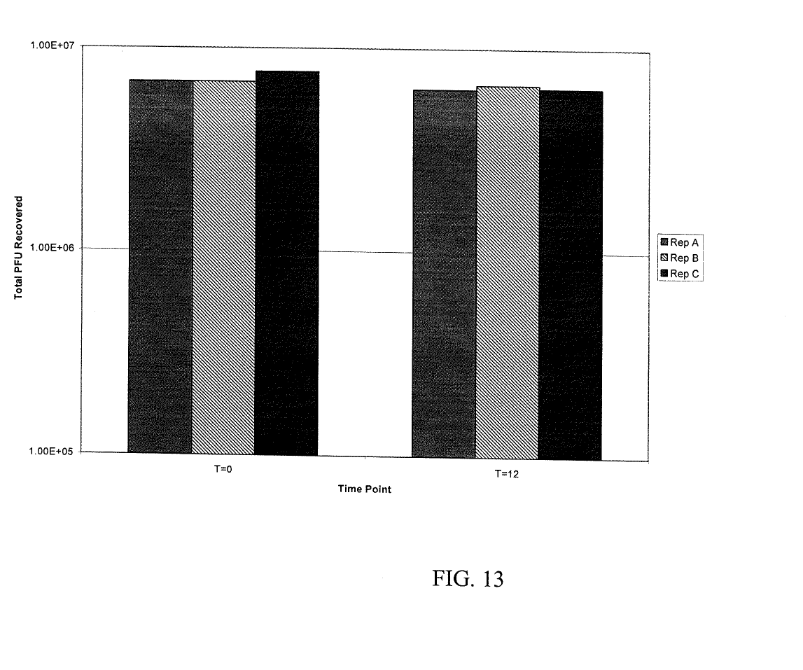

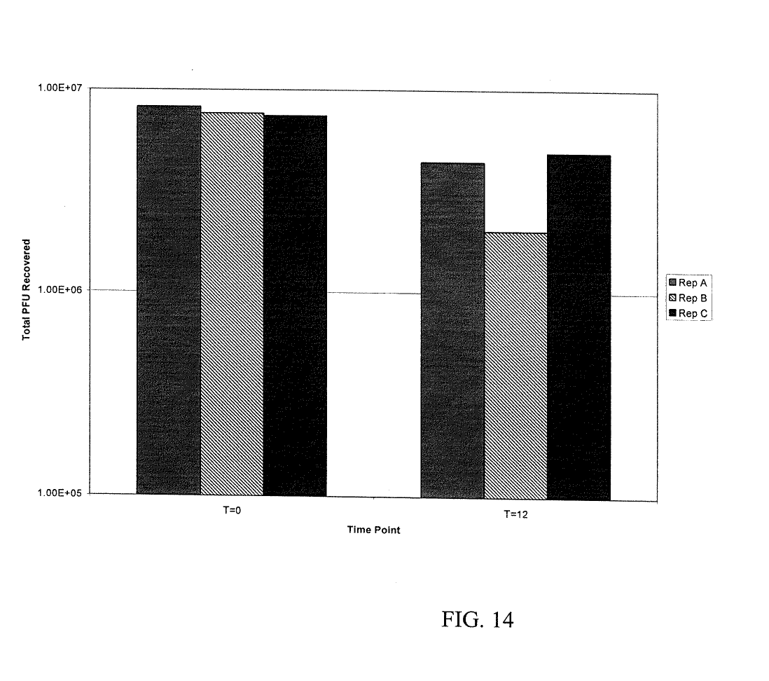

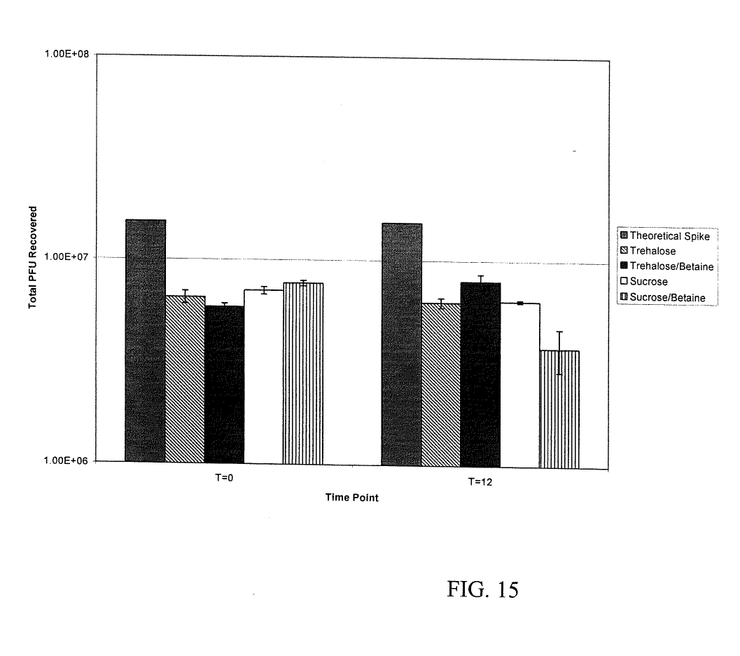

The results for all samples tested with MS2 can be seen in Table 3 and FIGS. 11-15. Table 3 presents the raw data counts as seen on the agar plates and also shows the calculated total virus recovery from each sample. FIGS. 11 through 14 compare the recoveries at each time point for each coating separately. According to these figures, recovery of virus appears to be consistent and reproducible regardless of time point or coating, except for T=12 on the sucrose/betaine coating. FIG. 15 summarizes FIGS. 11 through 14 and compares the virus recovery to the theoretical amount spiked onto the samples. FIG. 15 shows a slight loss in recovery of virus based on the theoretical amount spiked onto the samples, but there is no apparent difference in recovery between the two time points or the four different coatings. There may be a slight loss in recovery of virus at T=12 on the sucrose/betaine-coated samples.

TABLE 3
Raw Data - MS2 Bacteriophage

| | | | Dilution Plated | | | | | | | | | | |
|---|---|---|---|---|---|---|---|---|---|---|---|---|---|
| | | | 10⁻¹ | | | 10⁻² | | | 10⁻³ | | | | Recovery |
| TimePoint | Coating | Replicate | A | B | C | A | B | C | A | B | C | Avg | (total PFU) |
| 0 | T | A | NP | NP | NP | TNTC | TNTC | TNTC | 127 | 98 | 116 | 114 | 5.68E+06 |
| | | B | NP | NP | NP | TNTC | TNTC | TNTC | 135 | 137 | 143 | 138 | 6.92E+06 |
| | | C | NP | NP | NP | TNTC | TNTC | TNTC | 145 | 147 | 137 | 143 | 7.15E+06 |
| | T + B | A | NP | NP | NP | TNTC | TNTC | TNTC | 127 | 104 | 137 | 123 | 6.13E+06 |
| | | B | NP | NP | NP | TNTC | TNTC | TNTC | 108 | 103 | 117 | 109 | 5.47E+06 |
| | | C | NP | NP | NP | TNTC | TNTC | TNTC | 95 | 106 | 168 | 123 | 6.15E+06 |
| | S | A | NP | NP | NP | TNTC | TNTC | TNTC | 125 | 150 | 134 | 136 | 6.82E+06 |
| | | B | NP | NP | NP | TNTC | TNTC | TNTC | 147 | 146 | 116 | 136 | 6.82E+06 |
| | | C | NP | NP | NP | TNTC | TNTC | TNTC | 150 | 155 | 156 | 154 | 7.68E+06 |
| | S + B | A | NP | NP | NP | TNTC | TNTC | TNTC | 158 | 169 | NP | 164 | 8.18E+06 |
| | | B | NP | NP | NP | TNTC | TNTC | TNTC | 154 | 142 | 160 | 152 | 7.60E+06 |
| | | C | NP | NP | NP | TNTC | TNTC | TNTC | 148 | NP | NP | 148 | 7.40E+06 |
| 12 | T | A | NP | NP | NP | TNTC | TNTC | TNTC | 96 | 155 | 89 | 113 | 5.67E+06 |
| | | B | NP | NP | NP | TNTC | TNTC | TNTC | 142 | 138 | 130 | 137 | 6.83E+06 |
| | | C | NP | NP | NP | TNTC | TNTC | TNTC | 117 | 132 | 136 | 128 | 6.42E+06 |
| | T + B | A | NP | NP | NP | TNTC | TNTC | TNTC | 122 | 136 | 158 | 139 | 6.93E+06 |
| | | B | NP | NP | NP | TNTC | TNTC | TNTC | 195 | 155 | 125 | 158 | 7.92E+06 |
| | | C | NP | NP | NP | TNTC | TNTC | TNTC | 168 | 207 | 173 | 183 | 9.13E+06 |
| | S | A | NP | NP | NP | TNTC | TNTC | TNTC | 132 | 123 | 123 | 126 | 6.30E+06 |
| | | B | NP | NP | NP | TNTC | TNTC | TNTC | 126 | 150 | 121 | 132 | 6.62E+06 |
| | | C | NP | NP | NP | TNTC | TNTC | TNTC | 132 | 133 | 118 | 128 | 6.38E+06 |
| | S + B | A | NP | NP | NP | TNTC | TNTC | TNTC | 84 | 80 | 102 | 89 | 4.43E+06 |
| | | B | NP | NP | NP | TNTC | TNTC | TNTC | 39 | 44 | 39 | 41 | 2.03E+06 |
| | | C | NP | NP | NP | TNTC | TNTC | TNTC | 100 | 116 | 80 | 99 | 4.93E+06 |

T = 200 mM trehalose
T + B = 200 mM trehalose/2 mM betaine
S = 200 mM sucrose
S + B = 200 mM sucrose/2 mM betaine
NP = Not Plated
TNTC = Too Numerous To Count

### Example 4

In Example 4, ovalbumin was analyzed using an egg protein ELISA kit (Crystal Chem product number 140-OA). Recovered samples were diluted 1:5 in an extraction buffer containing 2-mercaptoethanol, followed by boiling the samples for five minutes. The samples were then further diluted in a diluent provided with the kit to the appropriate concentration to be analyzed on the ELISA 96-well plate following the instructions provided with the kit. The absorbance of the samples in each well was read at 450 nm. These values were compared to the standard curve and the concentrations of ovalbumin in each sample calculated.

### Example 4

### Results—Ovalbumin

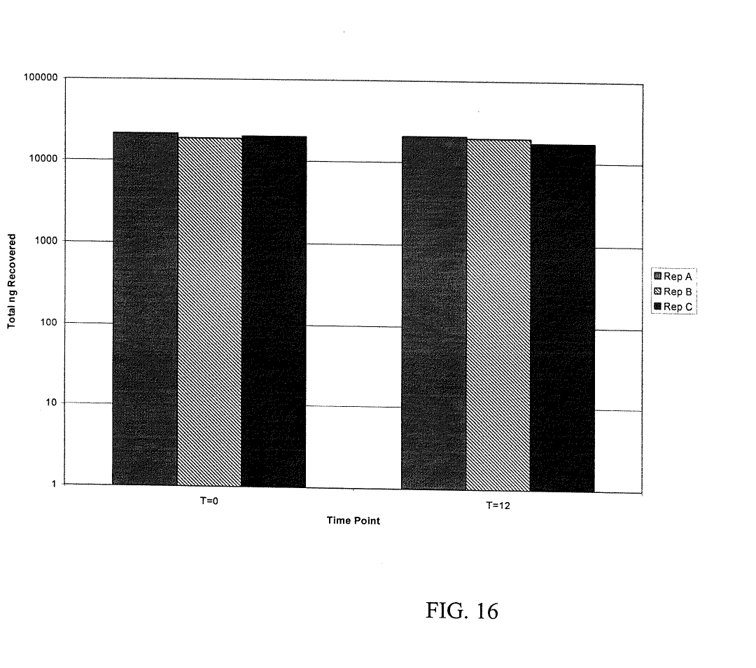

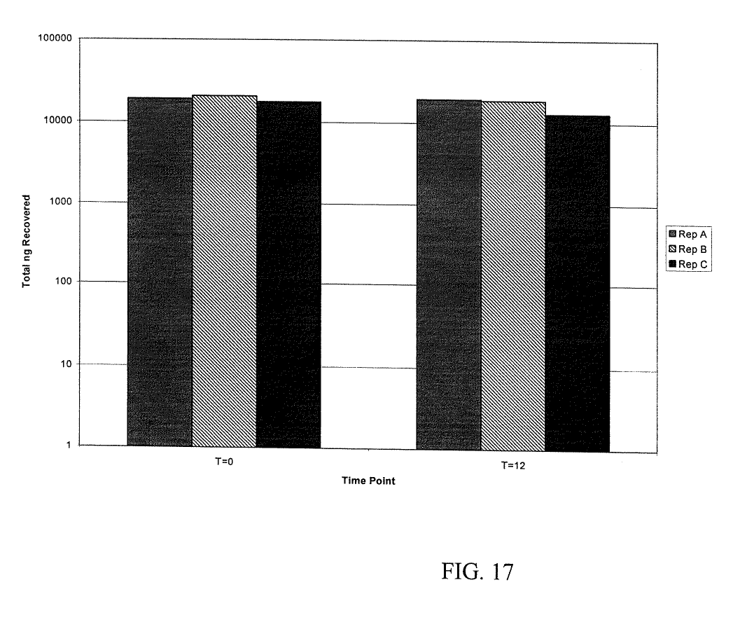

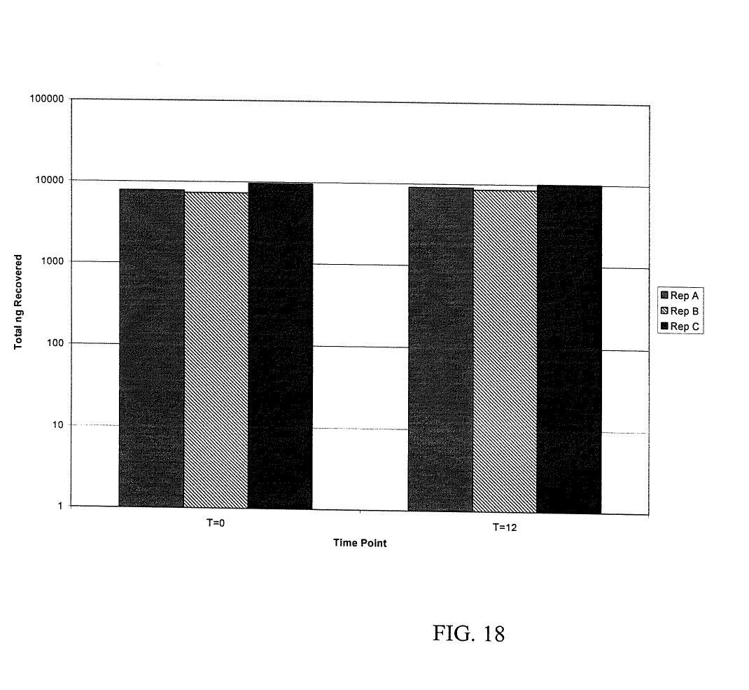

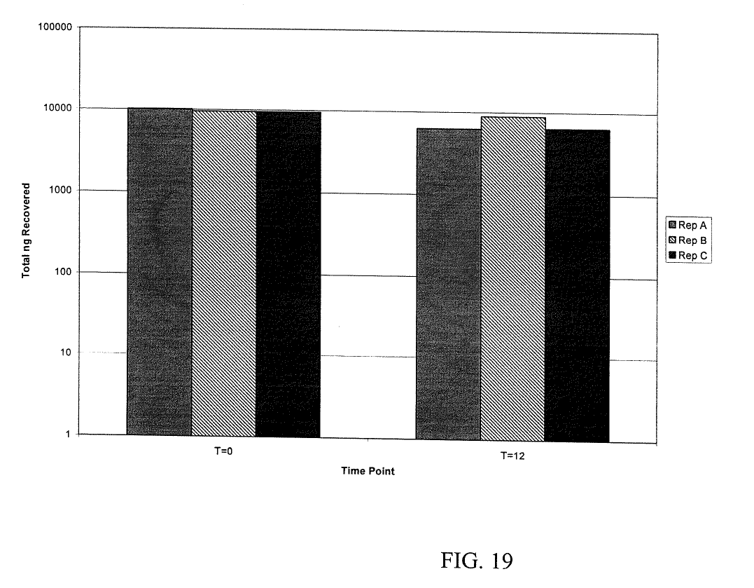

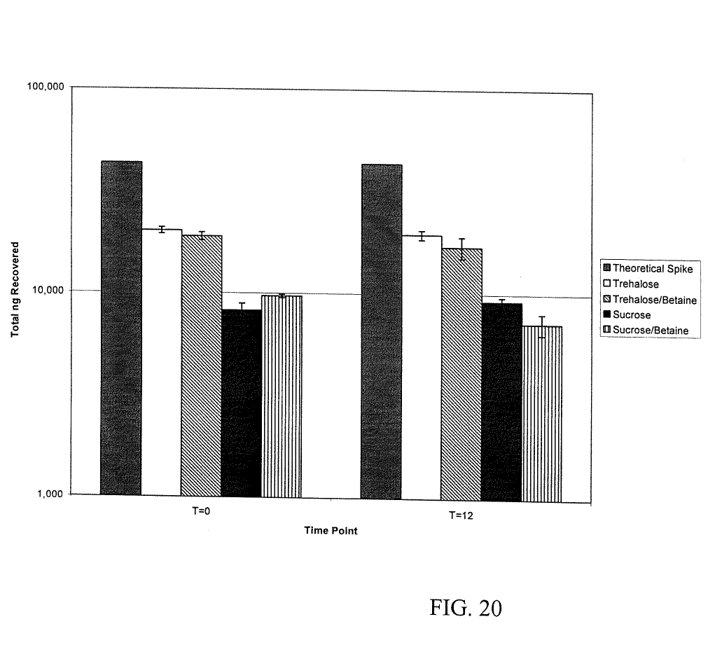

The results for all samples tested with ovalbumin can be seen in Table 4 and FIG. 16-20. Table 4 presents the raw ng/mL protein as detected on the ELISA and also shows the calculated total protein recovery from each sample. FIGS. 16 through 19 compare the recoveries at each time point for each coating separately. According to these figures, recovery of protein appears to be consistent and reproducible among the replicates regardless of time point or coating, although it appears recovery of protein from the sucrose and sucrose/betaine-coated tubes appears to be slightly less. FIG. 20 summarizes FIGS. 16 through 19 and compares the protein recovery to the theoretical amount spiked onto the samples. There is a slight loss in recovery of protein compared to the theoretical value spiked onto the tubes, but there appears to be no difference in recovery between the time points. There is a two-fold loss in recovery of protein from the tubes coated with sucrose solutions compared to those coated with trehalose solutions.

TABLE 4
Raw Data - Ovalbumin

| | | | Detected | | | | |
|---|---|---|---|---|---|---|---|
| | | | Ovalbumin | | | | |
| | | | Concentration | | | | |
| | | | (ng/mL) | | | | Total Recovered |
| TimePoint | Coating | Replicate | A | B | Avg | Dilution | Ovalbumin (ng) |
| 0 | T | A | 20.645 | 21.910 | 21.278 | 0.01 | 21278 |
| | | B | 18.703 | 18.849 | 18.776 | 0.01 | 18776 |
| | | C | 19.326 | 20.682 | 20.004 | 0.01 | 20004 |
| | T + B | A | 18.593 | 18.739 | 18.666 | 0.01 | 18666 |
| | | B | 20.224 | 20.315 | 20.270 | 0.01 | 20270 |
| | | C | 17.255 | 17.585 | 17.420 | 0.01 | 17420 |
| | S | A | 7.742 | 7.816 | 7.779 | 0.01 | 7779 |
| | | B | 7.284 | 7.339 | 7.312 | 0.01 | 7312 |
| | | C | 9.667 | 9.502 | 9.585 | 0.01 | 9585 |
| | S + B | A | 9.777 | 10.400 | 10.089 | 0.01 | 10089 |
| | | B | 9.245 | 9.868 | 9.557 | 0.01 | 9557 |
| | | C | 9.209 | 9.832 | 9.521 | 0.01 | 9521 |
| 12 | T | A | 21.250 | 20.297 | 20.774 | 0.01 | 20774 |
| | | B | 19.381 | 20.150 | 19.766 | 0.01 | 19766 |
| | | C | 16.925 | 17.768 | 17.347 | 0.01 | 17347 |
| | T + B | A | 18.629 | 19.564 | 19.097 | 0.01 | 19097 |
| | | B | 18.208 | 18.867 | 18.538 | 0.01 | 18538 |
| | | C | 12.966 | 12.911 | 12.939 | 0.01 | 12939 |
| | S | A | 8.952 | 9.172 | 9.062 | 0.01 | 9062 |
| | | B | 8.805 | 8.420 | 8.613 | 0.01 | 8613 |
| | | C | 10.363 | 9.777 | 10.070 | 0.01 | 10070 |
| | S + B | A | 6.056 | 6.496 | 6.276 | 0.01 | 6276 |
| | | B | 8.915 | 8.915 | 8.915 | 0.01 | 8915 |
| | | C | 6.459 | 6.367 | 6.413 | 0.01 | 6413 |

T = 200 mM trehalose
T + B = 200 mM trehalose/2 mM betaine
S = 200 mM sucrose
S + B = 200 mM sucrose/2 mM betaine

The coating solution for the capture and preservation of biological material is shown to aid in the collection and preservation of biological material. The coating solution can be used with a variety of existing collection devices. The coating solution is easy to prepare and the method for applying the coating solution to the collection device is easy and efficient. The coating solution is temperature insensitive, does not add weight to the collection device thereby making it easier to carry in the field, and cannot be spilled. If the collection device is kept on an airplane, fire truck, or other location susceptible to a bioterrism attack, these features become extremely important.

Having described the invention in detail, those skilled in the art will appreciate that modifications of the invention may be made without departing from the spirit and scope thereof. Therefore, it is not intended that the scope of the invention be limited to the specific embodiments and examples described. Rather, it is intended that the appended claims and their equivalents determine the scope of the invention.

We claim:

1. A coating solution for collecting and preserving biological material comprising:
from about 100 mM to 300 mM of at least one saccharide.

2. The coating solution of claim 1 wherein said saccharide is selected from the group consisting of glucose, fructose, galactose, ribose, trehalose, sucrose, lactose, maltose, cellobiose, raffinose, hydrophilic polysaccharides, and mixtures thereof.

3. The coating solution of claim 1 comprising from about 150 mM to 250 mM of said at least one saccharide.

4. The coating solution of claim 1 comprising about 200 mM of said at least one saccharide.

5. The coating solution of claim 1 further comprising at least one constituent selected from the group consisting of amino acids, complex proteins, surfactants, mixtures thereof, and derivatives thereof.

6. The coating solution of claim 5 further comprising from about 0.1 mM to 30 mM of said at least one constituent.

7. The coating solution of claim 5 further comprising from about 1 mM to 10 mM of said at least one constituent.

8. The coating solution of claim 5 further comprising about 2 mM of said at least one constituent.

9. The coating solution of claim 5 wherein said amino acid is selected from the group consisting of betaine, alanine, arginine, asparginine, aspartic acid, cysteine, glutamic acid, glutamine, glycine, histidine, isoleucine, leucine, lysine, methionine, phenylalanine, proline, serine, threonine, tryptophan, tyrosine, valine, mixtures thereof, and derivatives thereof.

10. The coating solution of claim 9 wherein said amino acid is betaine.

11. A coating solution for collecting and preserving biological material comprising:
about 200 mM of trehalose; and
about 2 mM of betaine.

12. A method of collecting and preserving biological material, comprising the steps of:
forming a coating solution including at least one saccharide;
applying said coating solution to a container;
curing said coating solution;
adding a biological material to said container;
dissolving said coating solution and said biological material in a liquid buffer; and
removing coating solution and said biological material.

13. The method of claim 12 wherein said coating solution further comprises at least one constituent selected from the group consisting of amino acids, complex proteins, surfactants, and mixtures thereof.

14. The method of claim 13 wherein said coating solution further comprises from about 0.1 mM to 30 mM of said constituent and from about 100 mM to 300 mM of said saccharide.

15. The method of claim 14 wherein said coating solution further comprises about 2 mM betaine and about 200 mM trehalose.

16. The method of claim 12 wherein said applying step further comprises the steps of:
partially filling said container with said coating solution;
coating said container evenly with said coating solution; and
removing any excess of said coating solution.

17. The method of claim 12 wherein said curing step further comprises the step of:
drying said container at a temperature of from about 59° F. to about 180° F.

18. The method of claim 12 wherein said curing step further comprises the step of:
drying said container in a vacuum oven at about 20 in. Hg vacuum and about 100° F.

19. The method of claim 12 wherein said liquid buffer solution comprises water and a substance selected from the group consisting of salts, surfactants, complex proteins and mixtures thereof.

20. The method of claim 12 wherein said liquid buffer comprises water, from about 0.1% to about 1.0% NaCl, and from about 0.01% to about 0.5% Triton X-100.

21. The method of claim 12 wherein said coating solution and said container are reusable.

22. The method of claim 12 wherein method further comprises the step of:
pre-treating said container prior to applying said coating solution to said container using a chemical, mechanical, or electrical treatment to prepare said container for application of said coating solution.

23. The method of claim 12 wherein said container includes a surface formed of a material selected from the group consisting of aluminum, alloys, metals, glass, filters, swabs, raw surfaces, plastics, composite laminates, fiberglass, painted surfaces, treated materials, pores materials, and semi-pores materials.
